(19) United States
(12) Patent Application Publication
(10) Pub. No.: US 2024/0285623 A1
(43) Pub. Date: Aug. 29, 2024

(54) TRPA1 CHANNEL ANTAGONIST COMPOUND FOR USE IN DEGENERATIVE RETINAL DISEASES

(71) Applicant: FLONEXT S.R.L., Firenze (IT)

(72) Inventors: Pierangelo GEPPETTI, Firenze (IT); Romina NASSINI, Firenze (IT); Francesco DE LOGU, Castiglione della Pescaia (GR) (IT)

(73) Assignee: FLONEXT S.R.L., Firenze (IT)

(21) Appl. No.: 18/567,877

(22) PCT Filed: Jun. 7, 2022
(86) PCT No.: PCT/IB2022/055276

§ 371 Date: Dec. 7, 2023

(30) Foreign Application Priority Data
Jun. 9, 2021 (IT) 102021000015098

(51) Int. Cl. A61K 31/506; A61K 31/416; A61K 31/4439; A61K 31/522; A61K 45/06; A61P 27/02

(52) U.S. Cl. CPC A61K 31/506; A61K 31/416; A61K 31/4439; A61K 31/522; A61K 45/06; A61P 27/02

(57) ABSTRACT

The present invention relates to TRPA1 channel antagonist compounds for use in the prevention and/or treatment of retinal diseases, in particular in the prevention and/or treatment of macular degeneration. The present invention also relates to an ophthalmic composition comprising at least one TRPA1 channel antagonist compound for topical ophthalmic use in the prevention and/or treatment of at least one degenerative retinal disease, preferably macular degeneration.

### FIELD OF THE INVENTION

The present invention relates to antagonist compounds of the transient receptor potential ankyrin 1 (TRPA1) channel for use in the prevention and/or treatment of retinal diseases, particularly in the prevention and/or treatment of macular degeneration. The present invention also relates to an ophthalmic composition comprising at least one TRPA1 channel antagonist compound for topical ophthalmic use in the prevention and/or treatment of at least one degenerative retinal disease, preferably macular degeneration.

### STATE OF THE ART

The retina is the transparent light-sensitive structure located in the ocular fundus. The central area of the retina, called macula, contains numerous photoreceptors, called cones, which are light-sensitive cells responsible for central and colour vision, while the rod cells, photoreceptors surrounding the macula, respond to lower light levels but are not sensitive to colours.

The retina can be affected by different types of pathologies which, depending on the retinal area affected, can have serious repercussions on vision.

Retinal damage can be direct or indirect. Among the pathologies with indirect retinal damage there is glaucoma. Glaucoma is an ocular disease due to increased pressure inside the eye which, in particular, occurs because the outflow pathways of the aqueous humour, the liquid that circulates inside the eye, ensuring nourishment to important ocular structures, become obstructed. The result is an increased ratio between aqueous humour produced and aqueous humour excreted and the pressure inside the bulb increases, exceeding the normal 14-16 mmHg. If this pressure increase is significant or lasts for a long time, it can damage the optic nerve. In addition to the damage to the optic nerve, the pathology is also characterized by alterations of the retinal nerve fiber layer, thus indirectly producing retinal damage as well.

Current medical therapy is essentially based on the use of eye drops with the function of reducing the production of aqueous humour or increasing its elimination. The first drug used was pilocarpine, an alkaloid from the plant Pilocarpus jaborandi, which is little used today due to some annoying side effects. The drugs currently used for the treatment of glaucoma are beta blockers, carbonic anhydrase inhibitors (including acetazolamide and diclofenamide), alpha stimulants, and prostaglandins (latanoprost).

Macular degeneration is one of the pathologies that directly damage the retina.

Macular degeneration is an age-related multifactorial disease affecting the macula. Macular degeneration is a progressive disease and is the main cause of irreversible blindness in adults over the age of 50. It is in fact a disease linked to aging and therefore destined to have an ever-wider impact on the world population due to increased life expectancy. Two different forms of age-related macular degeneration are known: the dry form (non-exudative or atrophic) and the wet form (exudative or neovascular). The dry form of age-related macular degeneration causes changes in the retinal pigment epithelium, which plays a critical role in keeping rods and cones healthy and well-functioning. The accumulation of waste products in cones and rods can lead to the formation of drusen, visible as yellow spots, which characterize the initial phase of age-related macular degeneration. The dry form is characterized by a progressive thinning of the central retina, which is poorly nourished by the capillaries and atrophies, resulting in the formation of an atrophic lesion in the macular area. Areas of chorioretinal atrophy (referred to as geographic atrophy) occur in more advanced cases of the dry form of age-related macular degeneration.

The wet form of macular degeneration, on the other hand, is characterized by the growth of abnormal blood vessels from the choroid, in correspondence with the macula (choroidal neovascularization). Focal macular oedema or hemorrhage can result in a raised macular area or a localized detachment of the retinal pigment epithelium. Finally, untreated neovascularization results in a submacular disciform scar. These newly formed blood vessels originate almost exclusively from the choroid (choroidal neovascularization) and cause the formation of a fibrovascular scar that destroys the central retina.

In general, wet macular degeneration, which is more aggressive than the dry form, can cause rapid and severe loss of central vision, caused by scarring of the blood vessels. Patients with the wet form of age-related macular degeneration show rapid loss of visual function, usually within days or weeks. The first symptom is generally visual distortion, characterized by the presence of scotomas or metamorphopsia (curvature of straight lines) secondary to the formation of new vessels near or in the center of the macula.

Most of the available treatments aim to prevent or cure the wet form of neovascular macular degeneration. However, there is still no established treatment for the dry form to date. Patients with extensive drusen, pigmentation changes, and/or geographic atrophy can reduce the risk of developing the advanced form of age-related macular degeneration by 25% by taking antioxidant vitamin and mineral supplements that generally include at least lutein or other vitamins, and sometimes zinc or other nutrients. Recently, omega-3 fatty acids have been prescribed to patients suffering from the dry form of age-related macular degeneration and included together with antioxidants among the dietary supplements on the market.

Such vitamin and antioxidant supplements can be used to soothe direct damage to the retina, especially direct degenerative damage, but not indirect damage. In fact, it has been observed that dietary supplements that can significantly reduce the risk of advanced macular degeneration have no benefit when used to treat glaucoma, as they do not reduce eye pressure. It is therefore evident that these two types of pathologies, while both involving the retina, have different molecular mechanisms. In fact, the damage caused by the excessive ocular pressure that characterizes glaucoma is localized at the level of the inner cells of the retina (ganglion, amacrine, horizontal, bipolar cells), but not of the retinal pigment epithelium (i.e. the RPE layer, from retinalpigment epithelium) and of photoreceptors (rods and cones). For the study of the two pathologies, namely glaucoma and macular degeneration, two animal models are used which present at the molecular level the typical lesions identified in a patient with glaucoma and in a patient with macular degeneration. The animal model of glaucoma presents a lesion localized in the internal cells of the retina (ganglion, amacrine, horizontal, bipolar cells) and does not affect the retinal pigment epithelium (Souza Monteiro de Araujo et al., 2020), whereas the animal model of macular degeneration shows damage at the central pole of the retina that can also extend to the peripheral area (Kiuchi, Current Eye Research 2002, Machalinska, Neurochemical Res. 2010, Wang, Invest Ophthalmol Vis Sci 2014, Commentaries NEURAL REGENERATION RESEARCH December 2014, Hanus, Cell Death Disc 2016, Chowers, Invest Ophtalmol 2017 and Koh, Journal of Photochemistry & Photobiology, 2019). Furthermore, it was observed that the loss of retinal pigment epithelium cells in the mouse model of macular degeneration affects not only the photoreceptors but also the underlying choriocapillaris layer. Damage to the choriocapillaris layer underlying the photoreceptors was not observed in the animal model of glaucoma. Due to the evident difference between these two pathologies that directly or indirectly affect the retina, the drugs used today for the treatment of glaucoma cannot be used for the treatment of macular degeneration, and in particular of dry macular degeneration, and vice versa.

Furthermore, with regards to macular degeneration, most of the treatments available aim to prevent or treat the wet form of macular degeneration but not the dry form for which, to date, there is still no established treatment.

The pharmacological treatment of choice for the wet form of macular degeneration includes the periodic administration by intravitreal injection of antagonist drugs of vascular endothelial growth factor (anti-VEGF) such as ranibizumab, bevacizumab, or aflibercept. In addition, corticosteroid drugs, such as triamcinolone, can be administered together with anti-VEGF drugs by intraocular injection. The intravitreal method of administration is an invasive route of administration that can lead to increased intraocular pressure, headache, vitritis (inflammation of the eye), vitreous detachment, retinal hemorrhage (bleeding from the back of the eye), visual disturbances and ocular pain. Intravitreal administration can also cause septic endophthalmitis, a serious intraocular inflammatory disease resulting from the infection of the vitreous cavity, which, although occurring infrequently (about 1/1000), can lead, in severe cases, to the loss of vision up to total blindness.

It is therefore evident that for the long-term treatment of age-related macular degeneration it is necessary to provide new, non-invasive therapies, which allow prevention and/or prolonged treatment without incurring side effects related to the method of administration.

### SUMMARY OF THE INVENTION

The Applicant addressed the problem of providing a new therapy for the prevention and/or prolonged treatment of degenerative retinal diseases, in particular macular degeneration, which does not involve the inconveniences and side effects of current therapies, in particular of therapies requiring intravitreal administration, and possibly of equal or greater efficacy.

The Applicant has surprisingly found that a new therapy based on the administration of at least one TRPA1 channel antagonist compound can be useful in the prevention and treatment of degenerative retinal diseases, such as macular degeneration.

Accordingly, a first aspect of the present invention is a TRPA1 channel antagonist compound for use in the prevention and/or treatment of at least one degenerative retinal disease selected from macular degeneration, diabetic retinopathy, retinal detachment, central serous chorioretinopathy, hypertensive retinopathy, macular hole, macular pucker, myodesopsia (floaters) and myopic maculopathy.

In particular, the Applicant observed that compounds acting as the TRPA1 channel antagonist compounds can be particularly effective in the prevention and/or treatment of macular degeneration.

Therefore, the TRPA1 channel antagonist compounds described in the present patent application can be advantageously used in the prevention and/or treatment of macular degeneration, and in particular, of both dry senile macular degeneration and wet senile macular degeneration.

A further aspect of the present invention is a pharmaceutical composition comprising at least one TRPA1 channel antagonist compound and at least one pharmaceutically acceptable excipient for use in the prevention and/or treatment of at least one degenerative retinal disease selected from macular degeneration, diabetic retinopathy, retinal detachment, central serous chorioretinopathy, hypertensive retinopathy, macular hole, macular pucker, myodesopsia (floaters) and myopic maculopathy.

Preferably, the pharmaceutical composition for use according to the invention is a topical ocular ophthalmic composition for use in the prevention and/or treatment of macular degeneration.

A further aspect of the present invention is a kit comprising the topical ocular ophthalmic composition, a container that contains it and a dispenser, where said topical ophthalmic composition is for use in the prevention and/or treatment of at least one degenerative retinal disease indicated above.

A further aspect of the present invention is a method of prevention and/or treatment of a degenerative retinal disease selected from macular degeneration, diabetic retinopathy, retinal detachment, central serous chorioretinopathy, hypertensive retinopathy, macular hole, macular pucker, myodesopsia (floaters), and myopic maculopathy, preferably of macular degeneration, which comprises administering to a patient at least one TRPA1 channel antagonist compound or the ophthalmic composition for use according to the invention.

The last aspect of the present invention is a combination of a TRPA1 channel antagonist compound and an anti-VEGF drug and/or a corticosteroid drug for simultaneous, separate or sequential use in the prevention and/or treatment of at least one degenerative retinal disease selected from macular degeneration, diabetic retinopathy, retinal detachment, central serous chorioretinopathy, hypertensive retinopathy, macular hole, macular pucker, myodesopsia (floaters) and myopic maculopathy.

### DETAILED DESCRIPTION OF THE INVENTION

A first aspect of the present invention is a TRPA1 channel antagonist compound for use in the prevention and/or treatment of at least one degenerative retinal disease.

The Applicant believes that the inhibition of the activity of the TRPA1 channel and of the signaling pathway downstream of it can prevent and/or treat degenerative retinal diseases.

The expression “TRPA1 channel antagonist” is intended to indicate a compound capable of exerting an inhibition activity of the TRPA1 channel and of the signaling pathway downstream of it. The compounds for use according to the invention act as TRPA1 channel antagonists as they inhibit its activity.

The TRPA1 receptor antagonist of the invention binds to the TRPA1 receptor with high affinity. In any case, the affinity of the binding of said TRPA1 antagonist is better than the binding affinity between said antagonist and another subtype of receptors of the TRP superfamily. Preferably, the binding affinity of said TRPA1 antagonist is at least 100 times higher than the binding affinity between said antagonist and another subtype of receptors of the TRP superfamily. Tests to determine whether a compound is a TRPA1 receptor antagonist are described in particular in Radresa et al. The Open Pain Journal 2013, 6, (Suppl1 M14) 137-153.

TRP channels represent a large and heterogeneous family of membrane ion channels permeable to monovalent and bivalent cations, in particular to sodium (Na⁺) and calcium (Ca²⁺) ions, largely involved in the modulation of the activation of sensory pathways. In mammals, 28 receptor subtypes have been identified, divided into six subfamilies: canonicals (TRPC1-7), vanilloids (TRPV1-6), ankyrins (TRPA), melastatin (TRPM1-8), polycystins (TRPP1-3) and mucolipins (TRPML1-3).

The TRPA1 channel is the only member of the ankyrin family.

In particular, the TRPA1 channel is expressed in the primary sensory neurons of the dorsal, trigeminal, nodular and vagal root ganglia, which give rise to afferent nerve fibers that transmit sensory signals of various kinds (mechanical, chemical and thermal).

The TRPA1 channel acts as a chemosensor of oxidative stress in the tissues subject to inflammation and plays a key role in signaling the pain stimulus.

The TRPA1 channel antagonist compound for use according to the invention can for example be selected from the compounds described in the articles Expert Opin. Ther. Patents 2012, 22, 663-95, Pharm. Pat. Anal. 2015, 4, 75-94 and Expert Opin. Ther. Patents 2020, 30, 643-657.

Examples of such compounds are also described, among others, in the articles Fanger et al.

TRPA1 as an Analgesic Target, The Open Drug Discovery Journal, 2010, 2, 64-70 and Chen et al. TRPA1 as a drug target—promise and challenges, Naunyn-Schmiedeberg's Arch Pharmacol, 2015, 388: 451-463 as well as in the patent documents reported herein and in the relative citations. The TRPA1 channel antagonist compound for use according to the invention can be prepared for example as described in the documents cited herein.

The TRPA1 channel antagonist compound for use according to the invention can be selected from compounds belonging to one of the following classes:
1) purinone derivatives and bioisosteres;
2) sulfonamide derivatives;
3) oxime derivatives;
4) amide derivatives;
5) polycyclic heteroaromatic derivatives;
6) indazole derivatives and bioisosteres;
7) phenylcarbamate derivatives and bioisosteres; or
8) decalin derivatives;
the salts thereof, optical isomers, solvates and prodrugs.

Preferably, the TRPA1 channel antagonist compound for use according to the invention can be selected from the compounds belonging to one of the following classes:
2) sulfonamide derivatives;
5) polycyclic heteroaromatic derivatives; or
6) indazole derivatives and bioisosteres;
the salts thereof, optical isomers, solvates and prodrugs.

The chemical classes of the compounds mentioned below are described below.

1) purinone derivatives and its bioisosteres, such as those shown among others in the documents:
WO2019152465A1 (Eli Lilly) relating to compounds of general formula

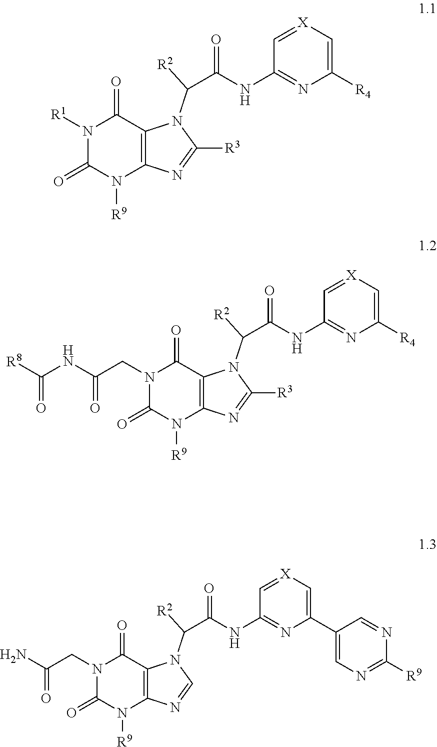

wherein the substituents have the meaning given in the document itself;
WO2015164643A1 (Hydra Biosciences) relating to compounds of general formula

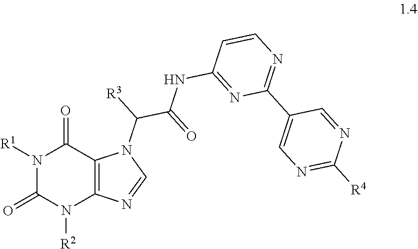

wherein the substituents have the meaning given in the document itself;
WO2016044792A1 (Hydra Biosciences) relating to compounds of general formula

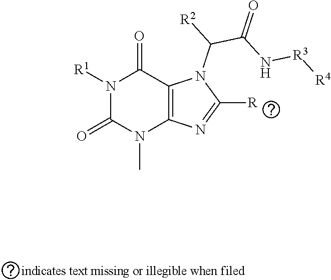

wherein the substituents have the meaning given in the document itself;
WO2015155306A1 (Almirall) relating to compounds of general formula

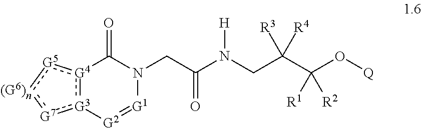

wherein the substituents have the meaning given in the document itself;
WO2017060488A1 (Almirall) relating to compounds of general formula

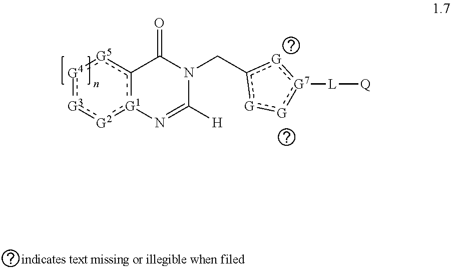

wherein the substituents have the meaning given in the document itself;
WO2017064068A1 (Almirall) relating to compounds of general formula

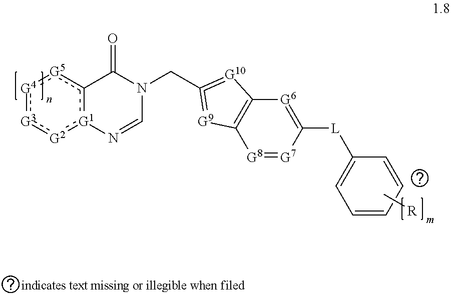

wherein the substituents have the meaning given in the document itself;
WO2015056094A2 and WO2016042501A1 (Glenmark) relating to compounds of general formula

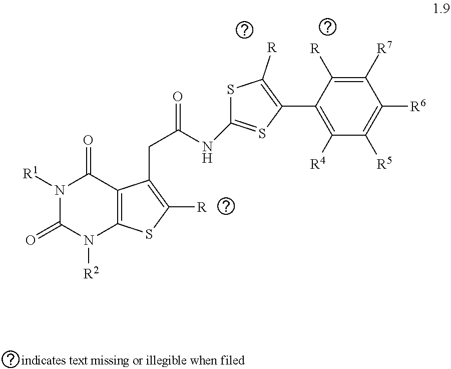

wherein the substituents have the meaning given in the document itself;
J. Med. Chem. 2016, 59, 2794-2809 (Amgen) relating to compound AM-0902 (CAS No. 1883711-97-4) of formula

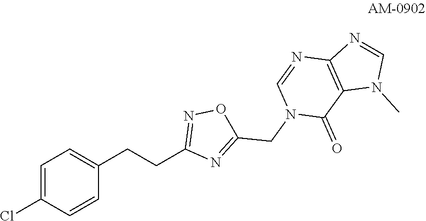 [SMILES: Cn1cnc2ncn(Cc3nc(CCc4ccc(Cl)cc4)no3)c(=O)c21]

WO2018096159A1 (Hoffmann La Roche, Genentech) relating to compounds of general formula

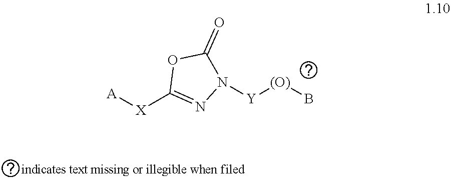

wherein the substituents have the meaning given in the document itself; WO2018162607A1 (Hoffmann La Roche, Genentech) relating to compounds of general formula

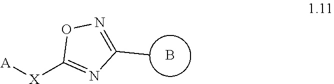

wherein the substituents have the meaning given in the document itself; WO2019182925A1 (Hoffmann La Roche, Genentech) relating to compounds of general formula

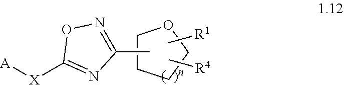

wherein the substituents have the meaning given in the document itself;
WO2021074198A1 (Boheringer) relating to tetrazole derivatives selected from the list consisting of nine compounds of claim 1 (list 1.13);
WO2013023102A1 (Hydra Biosciences) relating to compounds of general formula

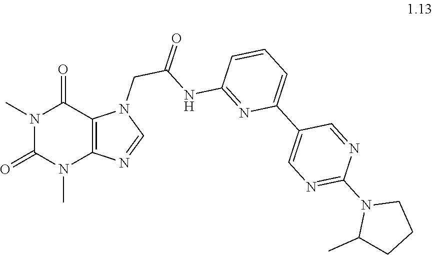

wherein the substituents have the meaning given in the document itself.

Preferred compounds of this class are the compounds
HC-030031 (Hydra Biosciences, CAS No. 349085-38-7) of formula

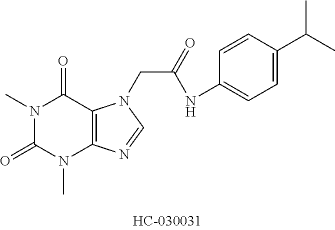 [SMILES: CC(C)c1ccc(NC(=O)Cn2cnc3c2c(=O)n(C)c(=O)n3C)cc1]

and similar shown in WO2012050641A1;
Chembridge-5861528 (Alomone Labs, CAS No.: 332117-28-9) of formula

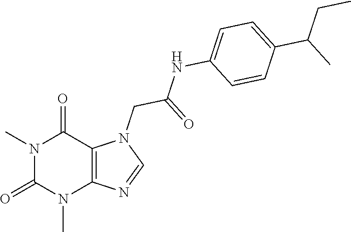 [SMILES: CCC(C)c1ccc(NC(=O)Cn2cnc3c2c(=O)n(C)c(=O)n3C)cc1]

CB-189625 and HX-100 (Hydra Biosciences and Cubist Pharmaceuticals) and similar of formula

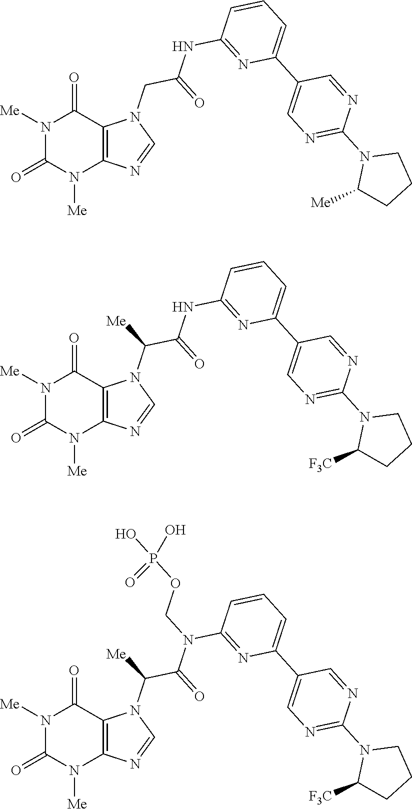 [SMILES: C[C@H]1CCCN1c1ncc(-c2cccc(NC(=O)Cn3cnc4c3c(=O)n(C)c(=O)n4C)n2)cn1 ; C[C@@H](C(=O)Nc1cccc(-c2cnc(N3CCC[C@H]3C(F)(F)F)nc2)n1)n1cnc2c1c(=O)n(C)c(=O)n2C ; C[C@@H](C(=O)N(COP(=O)(O)O)c1cccc(-c2cnc(N3CCC[C@H]3C(F)(F)F)nc2)n1)n1cnc2c1c(=O)n(C)c(=O)n2C]

GRC 17536 (Glenmark Pharmaceuticals, CAS No: 1649479-05-9) of formula

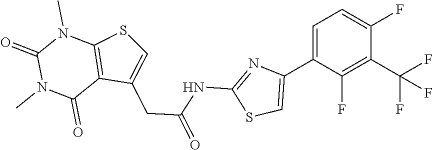 [SMILES: Cn1c(=O)c2c(CC(=O)Nc3nc(-c4ccc(F)c(C(F)(F)F)c4F)cs3)csc2n(C)c1=O]

2) sulfonamide derivatives such as those shown among others in the documents:
WO2014049047A1 (Hoffmann La Roche) relating to compounds of general formula

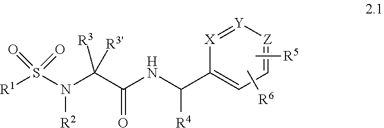

wherein the substituents have the meaning given in the document itself;
WO2015052264A1 (Hoffmann La Roche, Genentech) relating to compounds of general formula

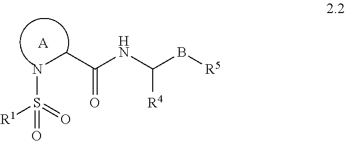

wherein the substituents have the meaning given in the document itself;
WO2016128529A1 (Hoffmann La Roche, Genentech) relating to compounds of general formula

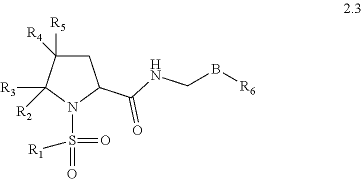

wherein the substituents have the meaning given in the document itself;
WO2018015410A1 (Hoffmann La Roche, Genentech) relating to compounds of general formula

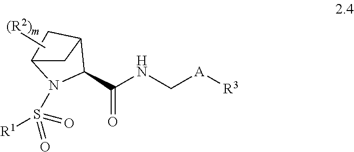

wherein the substituents have the meaning given in the document itself; WO2018029288A1 (Hoffmann La Roche, Genentech) relating to compounds of general formula

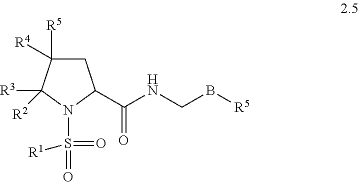

wherein the substituents have the meaning given in the document itself;
WO2015115507 A1 (Ajinomoto) relating to compounds of general formula

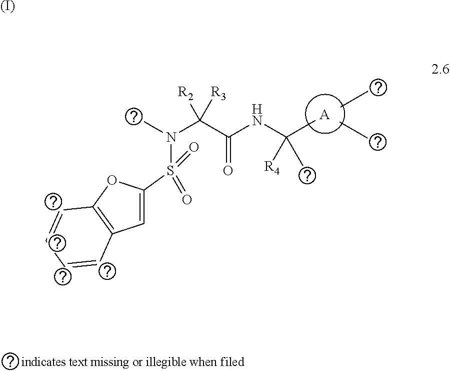

wherein the substituents have the meaning given in the document itself;
WO2017018495A1 (EA Pharma) relating to compounds of general formula

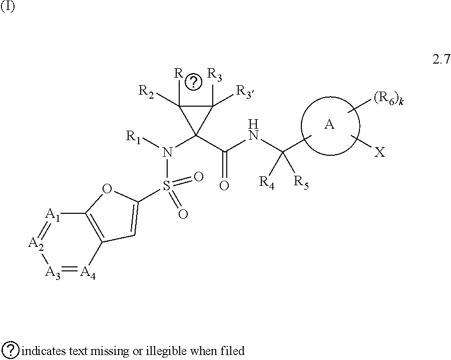

wherein the substituents have the meaning given in the document itself;
WO2017135462A1 (EA Pharma) relating to compounds of general formula

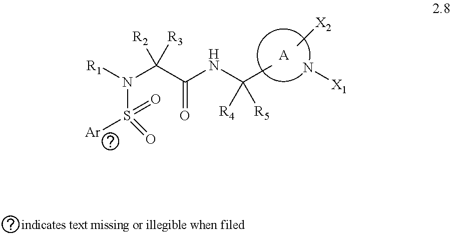

wherein the substituents have the meaning given in the document itself;
WO2010141805A1 (Janssen) relating to compounds of general formula

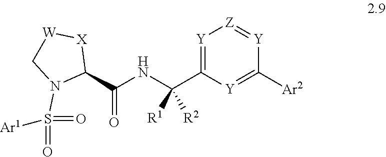

wherein the substituents have the meaning given in the document itself;
WO2012152983 (Orion) relating to compounds of general formula

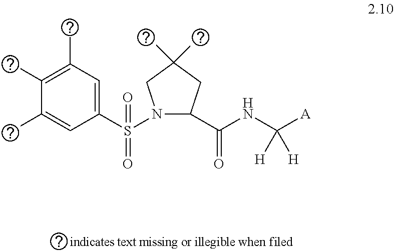

wherein the substituents have the meaning given in the document itself;
EP2805718 (WO2013108857A1, Ajinomoto) relating to compounds of general formula

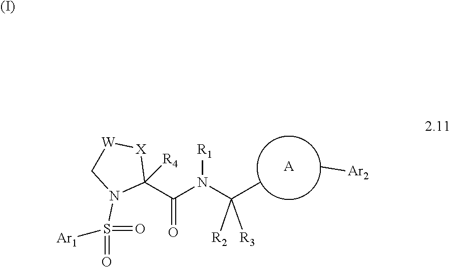

wherein the substituents have the meaning given in the document itself.

According to a preferred embodiment, the compounds of this class are represented by the following general formula A1

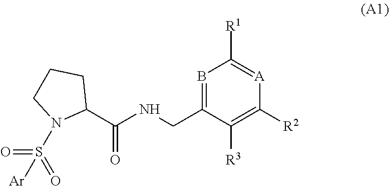

where A and B, equal or different from each other, can represent a CH group or a nitrogen atom, Ar can represent a 5 or 6-membered aromatic cycle, preferably selected from the group consisting of aryl, pyridine, pyrimidine, pyrazine, pyrrole, imidazole, furan, thiophene, and thiazole, optionally substituted with one or more halogen atoms, preferably with one or more fluorine or chlorine atoms, and R1, R2 and R3 equal or different from each other can represent a hydrogen atom, a fluoromethyl group, or the residue of formula:

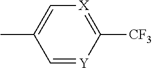

where X and Y, equal or different from each other, can represent a CH group or a nitrogen atom.

Preferred compounds of this class are the compounds
JNJ-41477670 (Janssen) of formula

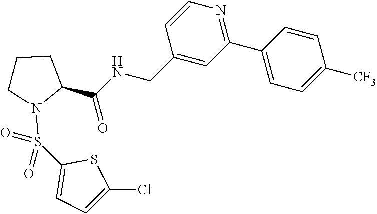 [SMILES: O=C(NCc1ccnc(-c2ccc(C(F)(F)F)cc2)c1)[C@@H]1CCCN1S(=O)(=O)c1ccc(Cl)s1]

Janssen

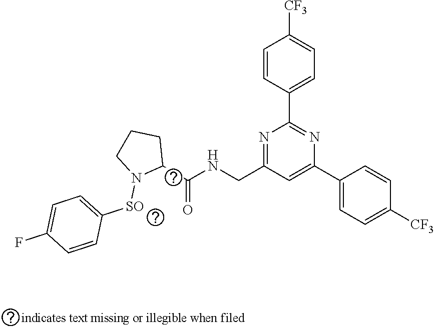

GDC-0334 (Genentech/Roche) of formula (IV)

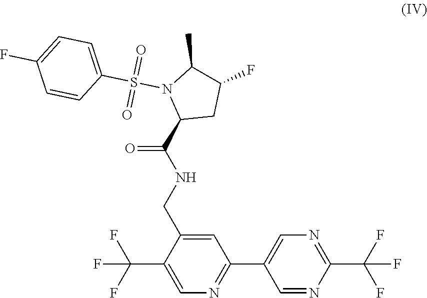 [SMILES: C[C@H]1[C@H](F)C[C@@H](C(=O)NCc2cc(-c3cnc(C(F)(F)F)nc3)ncc2C(F)(F)F)N1S(=O)(=O)c1ccc(F)cc1]

In a particularly preferred embodiment, the compound for use in the prevention and/or treatment of at least one degenerative retinal disease is the compound of formula (IV).

In particular, the compound of formula (IV) is able to prevent and/or treat macular degeneration, both dry senile macular degeneration and wet senile macular degeneration. In fact, as shown in the experimental section, the compound of formula (IV) proved to be particularly effective in protecting the retina from NalO₃-induced damage of the cells of the RPE layer, which is of fundamental importance for maintaining the function of the photoreceptors of the macula.

3) oxime derivatives, such as those shown among others in the documents:
WO2009089083A1 (Abbott) relating to compounds of general formula

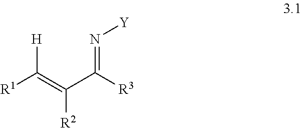

wherein the substituents have the meaning given in the document itself;
WO2009089082A1 (Abbott) relating to compounds of general formula

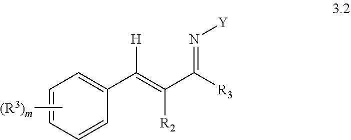

wherein the substituents have the meaning given in the document itself.

Preferred compounds of this class are compounds
A967079 (Abbott) of formula

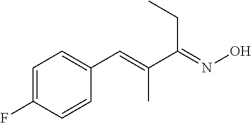

described in the document Pain 2011, 152, 1165-1172;
AP-18 (CAS No. 55224-94-7) of formula

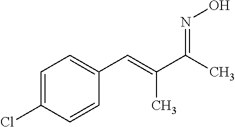

4) amide derivatives, such as those shown among others in the documents:
WO2016067143A1 (Pfizer) relating to compounds of general formula

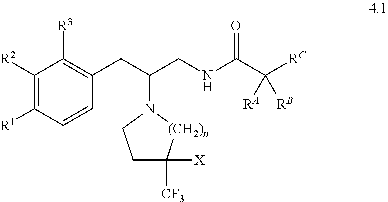

in particular, the compound of formula

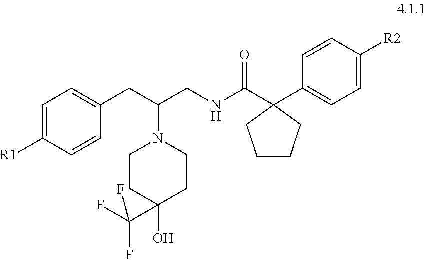

wherein the substituents have the meaning given in the document itself;
WO2014053694A1 (Orion Corporation) relating to compounds of general formula

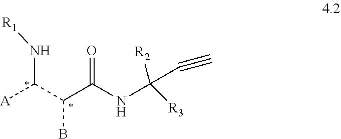

in particular, of the pyridine-3-carboxamide of formula

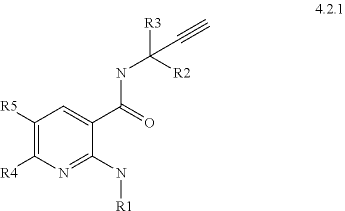

wherein the substituents have the meaning given in the document itself;
WO2015144976A1 (Orion Corporation) relating to compounds of general formula

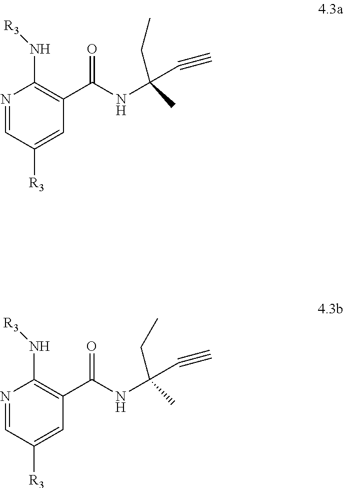

wherein the substituents have the meaning given in the document itself;
WO2015144977A1 (Orion Corporation) relating to compounds of general formula

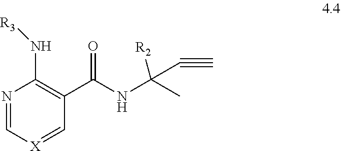

wherein the substituents have the meaning given in the document itself;
a compound of Orion Pharma of particular interest is the one identified by the acronym ODM-108.
WO2012050512A1 (ASTRAZENECA) relating to compounds of general formula

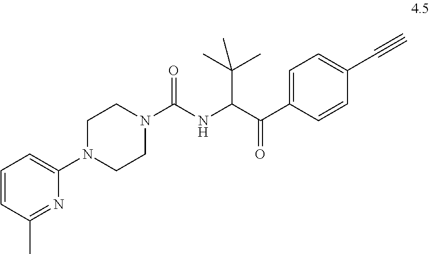

wherein the substituents have the meaning given in the document itself; WO2020244460A1 (HANGZHOU WESTAN PHARMACEUTICAL) relating to compounds of general formula

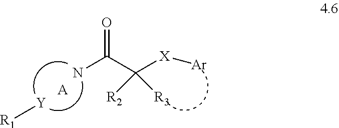

wherein the substituents have the meaning given in the document itself;
WO2018015411A1 (Hoffmann La Roche, Genentech) relating to compounds of general

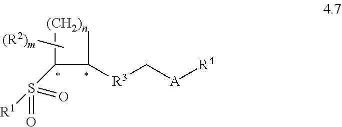

wherein R³ is —NHCO— or —CONH— and the other substituents have the meaning given in the document itself.

Preferred compounds of this class are the following compounds:
AZ465 (ASTRAZENECA) of formula

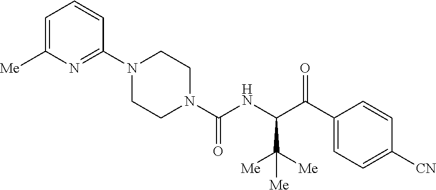 [SMILES: Cc1cccc(N2CCN(C(=O)N[C@@H](C(=O)c3ccc(C#N)cc3)C(C)(C)C)CC2)n1]

Pfizer of formula

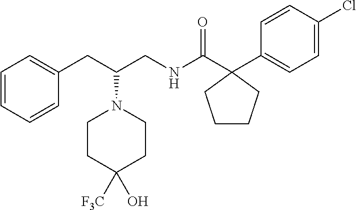

Orion of formula

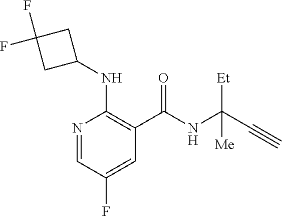 [SMILES: C#CC(C)(CC)NC(=O)c1cc(F)cnc1NC1CC(F)(F)C1]

5) polycyclic heteroaromatic derivatives such as those shown among others in the documents:
WO2015103060A1 and WO2018009717A1 (Algomedix) relating to compounds of general formula

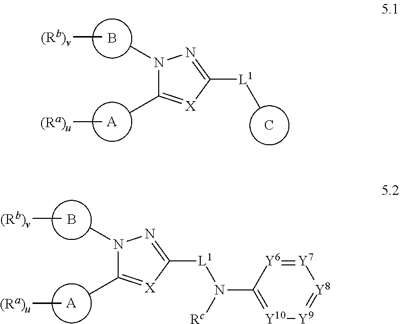

wherein the substituents have the meaning given in the document itself;
Bioorg. Med. Chem. Lett. 2014, 24, 3464-3468 (Amgen) relating to compounds of general formula

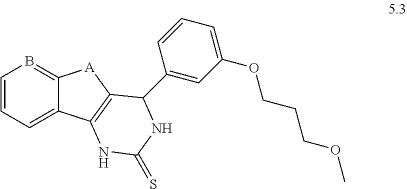

wherein the substituents have the meaning given in the document itself; WO2009147079A1 (Janssen) relating to compounds of general formula

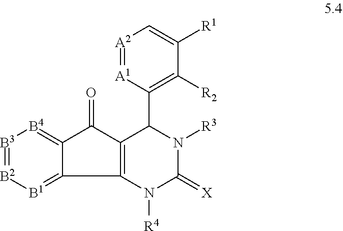

wherein the substituents have the meaning given in the document itself.

According to a preferred embodiment, the compounds of this class are represented by the following general formula A2

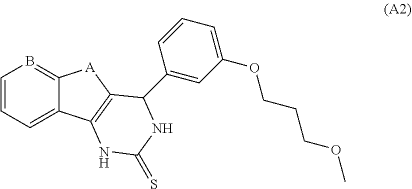

where A can represent an oxygen atom, a —NH— group, or a carbonyl group —(C═O)—, and B can represent a —CH— group or a nitrogen atom.

Preferred compounds of the class of polycyclic heteroaromatic derivatives are the compounds of formula (I) and (II) reported below:

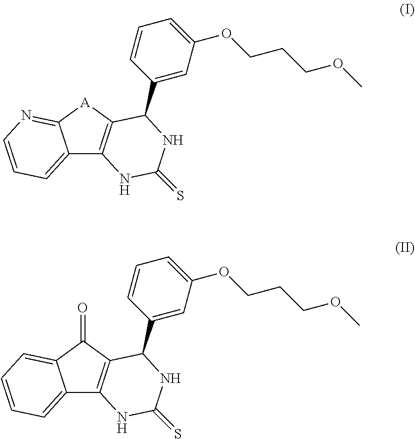

The compound of formula (I) is a TRPA1 channel antagonist developed by Amgen (Compound 10, CAS No 1620518-03-7).

The compound of formula (II) is a TRPA1 channel antagonist developed by Janssen (Compound 43, CAS No 1198174-47-8).

In a particularly preferred embodiment, the compound for use according to the invention is the compound of formula (II).

In particular, the compound of formula (II) is able to prevent and/or treat macular degeneration, both dry age-related macular degeneration and wet age-related macular degeneration. In fact, as shown in the experimental section, the compound of formula (II) proved to be particularly effective in protecting the retina from NalO₃-induced damage of the cells of the RPE layer, which is of fundamental importance for maintaining the function of the photoreceptors of the macula.

In a further particularly preferred embodiment, the compound for use according to the invention is the compound of formula (I).

In particular, the compound of formula (I) is able to prevent and/or treat macular degeneration, both dry age-related macular degeneration and wet age-related macular degeneration.

In fact, as shown in the experimental section, the compound of formula (I) proved to be particularly effective in protecting the retina from NalO₃-induced damage of the cells of the RPE layer, which is of fundamental importance for maintaining the function of the photoreceptors of the macula.

6) indazole derivatives and bioisosteres such as those shown among others in the documents:
J. Med. Chem. 2014, 57, 5129-5140 (Novartis) relating to compounds of general formula

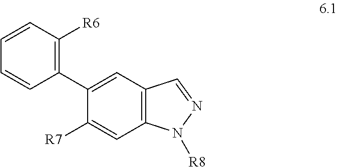

wherein the substituents have the meaning given in the document itself;
ACS Med Chem Lett. 2017; 8, 666-671 (Pfizer, amino and aryl indazoles, Tables 1 and 2);
According to a preferred embodiment, the compounds of this class are represented by the following general formula A3:

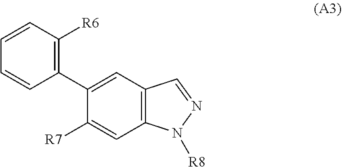

where R6, R7 and R8, equal or different from each other, can represent a hydrogen atom, an alkyl group having from 1 to 3 carbon atoms, or a trifluoromethyl group.

The preferred compound of this class is given below:

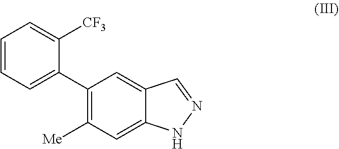 [SMILES: Cc1cc2[nH]ncc2cc1-c1ccccc1C(F)(F)F]

The compound of formula (III) is a TRPA1 channel antagonist developed by Novartis (CAS No 1613505-14-8).

In a particularly preferred embodiment, the compound for use according to the invention is the compound of formula (III).

In particular, the compound of formula (III) is able to prevent and/or treat macular degeneration, both dry senile macular degeneration and wet senile macular degeneration.

In fact, as shown in the experimental section, the compound of formula (III) proved to be particularly effective in protecting the retina from NalO₃-induced damage of the cells of the RPE layer, which is of fundamental importance for maintaining the function of the macula photoreceptors.

7) phenylcarbamate derivatives and bioisosteres, such as those shown among others in the documents:
WO2014056958A1 (Hofmann—La Roche) relating to compounds of general formula

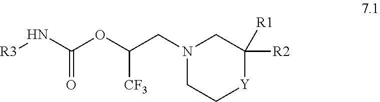

wherein the substituents have the meaning given in the document itself;
WO2014060341A1 (Hofmann—La Roche) relating to compounds of general formula

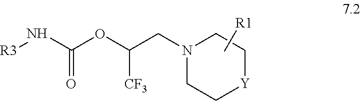

wherein the substituents have the meaning given in the document itself;
WO2014072325A1 (Hofmann—La Roche) relating to compounds of general formula

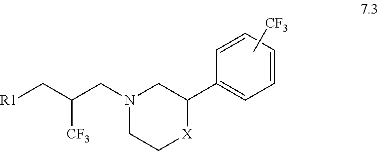

wherein the substituents have the meaning given in the document itself.

Preferred compounds of this class are the compounds
Hofmann—La Roche

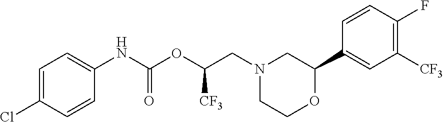 [SMILES: O=C(Nc1ccc(Cl)cc1)O[C@@H](CN1CCO[C@H](c2ccc(F)c(C(F)(F)F)c2)C1)C(F)(F)F]

8) decalin derivatives such as those shown among others in the documents:
WO2011043954A1 (Merck) relating to compounds of general formula

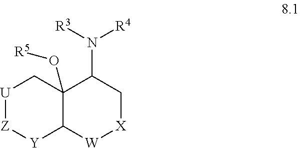

in particular, the compounds of formula

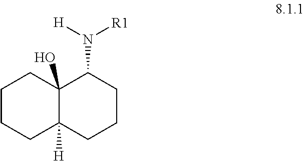

wherein the substituents have the meaning given in the document itself;
Preferred compounds of this class are the compounds

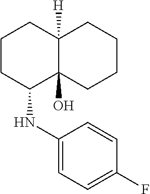 [SMILES: O[C@]12CCCC[C@@H]1CCC[C@H]2Nc1ccc(F)cc1]

The TRPA1 channel antagonist compound for use according to the present invention can be in the form of salt, optical isomer, pure or in a mixture, solvate or pro-drug, provided that it is pharmaceutically acceptable.

The term “pro-drug” refers to a biologically inactive molecule which, once introduced into the body, undergoes chemical transformations by enzymes that activate it. The prodrug is therefore a precursor to the active ingredient.

Preferably, the compound for use according to the invention is selected from the compounds belonging to the class of polycyclic derivatives or indazole derivatives, since they are characterized by good pharmacokinetic properties and a better action profile.

The TRPA1 channel antagonist compounds, preferably belonging to the class of polycyclic derivatives or indazole derivatives, are able to prevent and/or treat at least one degenerative retinal disease selected from macular degeneration, diabetic retinopathy, retinal detachment, serous chorioretinopathy central, hypertensive retinopathy, macular hole, macular pucker, myodesopsia (floaters) and myopic maculopathy.

According to a preferred aspect of the invention, the compounds of the invention, preferably belonging to the class of polycyclic derivatives or indazole derivatives, are able to prevent and/or treat a particular type of degenerative retinal disease: macular degeneration. Macular degeneration falls into the macro category of maculopathies.

In the present invention, the term “macular degeneration” is used to indicate a particular type of degenerative maculopathy.

The term “maculopathy” refers to a pathology that affects the central part of the retina, called macula. Maculopathies can be classified into acquired maculopathies, myopic maculopathies and hereditary maculopathies. According to another aspect of the invention, the TRPA1 channel antagonist compounds can be used for the prevention and/or treatment of myopic maculopathy. Myopic maculopathy occurs in people with degenerative or pathological myopia. In particular, in subjects suffering from myopic maculopathy, the retina is unable to adapt to the elongation of the bulb and suffers injuries. In pathological myopia, macular hemorrhages can occur with a sudden decrease in visual acuity, sometimes with image distortion.

The most common acquired maculopathy is age-related macular degeneration.

Macular degeneration is a disease characterized by the deterioration of the macula, the central portion of the retina responsible for central vision.

In the present invention, the expressions “age-related macular degeneration” and “senile macular degeneration” both indicate the retinal degenerative maculopathy described above. According to the present invention, the TRPA1 channel antagonist compound as defined above is useful for the prevention and/or treatment of both forms of macular degeneration, namely dry senile macular degeneration and wet senile macular degeneration.

A further aspect of the present invention relates to a pharmaceutical composition, preferably an ophthalmic composition, more preferably an ocular topical ophthalmic composition, comprising a therapeutically effective amount of at least one TRPA1 channel antagonist compound and at least one pharmaceutically acceptable excipient for use in the prevention and/or treatment of at least one degenerative retinal disease selected from macular degeneration, diabetic retinopathy, retinal detachment, central serous chorioretinopathy, hypertensive retinopathy, macular hole, macular pucker, myodesopsia (floaters), and myopic maculopathy, preferably of macular degeneration in both of its forms.

In the pharmaceutical composition for use according to the invention, said at least one TRPA1 channel antagonist compound belongs to one of the classes from 1) to 8) described above, preferably to classes 2) 5) and 6), more preferably to classes 5) and 6), respectively of polycyclic heteroaromatic derivatives or indazole derivatives and bioisosteres.

In a particularly preferred embodiment, the compound belonging to the class of sulfonamide derivatives has the general formula A1) indicated above. More preferably, said compound belonging to the class of sulfonamide derivatives and having general formula A1) is the compound of formula (IV) indicated above.

In a particularly preferred embodiment, the compound belonging to the class of polycyclic derivatives present in said pharmaceutical composition has general formula A2), said compound being preferably selected from the compound of formula (I) or (II) indicated above.

In a particularly preferred embodiment, the compound belonging to the class of indazole derivatives and bioisosteres present in said pharmaceutical composition has general formula A3.

More preferably, said compound belonging to the class of indazole derivatives and bioisosteres and having general formula A3 is the compound of formula (III) indicated above. According to one aspect, the ophthalmic composition comprises a plurality of TRPA1 channel antagonist compounds, preferably at least two TRPA1 channel antagonist compounds, said TRPA1 channel antagonist compounds preferably belonging to the class of polycyclic derivatives and/or indazole derivatives, preferably selected from the compounds of formula (I) or (II) and (III) indicated above, even more preferably between the compounds of formula (I) and (III).

The pharmaceutical composition, preferably ophthalmic, for use according to the invention, can be advantageously used for the prevention and/or treatment of degenerative retinal diseases, preferably selected from macular degeneration, diabetic retinopathy, retinal detachment, central serous chorioretinopathy, hypertensive retinopathy, macular hole, macular pucker, myodesopsia (floaters) and myopic maculopathy.

Preferably, said retinal degenerative pathology is macular degeneration, in particular dry senile macular degeneration and wet senile macular degeneration.

The pharmaceutical composition for use according to the invention can be administered according to any route of administration, as long as it is suitable for achieving concentrations at the retinal level that are effective for the prevention or treatment of the pathology in question. Preferably the pharmaceutical composition for use according to the invention is an ophthalmic composition, suitable for being administered internally or externally to the eye.

According to one embodiment, the present composition is a composition suitable for administration to the posterior segment of the eye, for example by injection or surgical implant, in particular suitable for administration to the retina, sclera, posterior chamber, vitreous chamber, subretinal space or to the suprachoroidal segment of the eye.

According to another preferred embodiment, the present composition is a composition suitable for administration to the anterior segment of the eye, by injection or surgical implant, in particular suitable for administration to the retina, sclera, posterior chamber, vitreous chamber, subretinal space or to the suprachoroidal segment of the eye.

In another more preferred embodiment, the composition for use according to the invention is a topical ophthalmic composition, suitable for being administered externally to the eye, for example by application in the lower eyelid pouch or conjunctival fornix, on the outer surface of the cornea.

The topical ophthalmic composition for use according to the invention can be formulated, for example, in the form of a solution, suspension, emulsion, gel, ointment, eye insert or therapeutic contact lens. The topical use of the composition of the invention, for example in the form of drops or eye drops, advantageously allows treating in a non-invasive way one or more retinal diseases, in particular macular degeneration, and avoids the inconveniences and side effects of intravitreal administration, which is commonly used today for the treatment of macular degeneration.

The ophthalmic composition for use according to the invention can comprise one or more ophthalmologically acceptable additives and/or excipients selected from those commonly used for ophthalmic formulations.

An “ophthalmologically acceptable excipient” is an inert excipient which allows the administration of a medicament to the eye and/or eyelids, to treat an ocular disease or condition without exerting deleterious effects on the eye. Generally, these are substances that contribute to increasing the efficacy and tolerability of the products where they are contained, as well as favoring their conservation over time.

Examples of said ophthalmologically acceptable additives or excipients are viscosifiers, permeation enhancers, buffering agents, osmolarity regulators, antioxidants, preservatives and surfactants.

Viscosifiers, which have the function of increasing the viscosity of the composition and consequently the contact time of the drug with the ocular surfaces, are preferably selected from cellulose derivatives, preferably hydroxymethyl cellulose, hydroxyethyl cellulose, hydroxypropyl methylcellulose, methylcellulose; polyethylene glycol, polyvinylpyrrolidone, polyvinylacetic alcohol, dextran, gelatin, glycerin, polysorbate 80 and other gelling agents.

Permeation enhancers, which have the function of increasing the permeability of the drug across the ocular membranes, are preferably selected from cyclodextrins, chelating agents, corona ethers, bile acids and bile salts.

Buffering agents have the function of supplying and maintaining the pH of the composition as close as possible to the physiological one, preferably between 6 and 8. This action is essential to allow good tolerability of the preparations and to maintain their efficacy. The preferred buffer is phosphate buffer, but other buffers capable of maintaining the pH within the desired range are also included, as long as they are suitable for ophthalmic use.

Osmolarity regulators are salts capable of making the liquid composition isotonic with ocular fluids. The preferred salt is sodium chloride (NaCl), but other biologically acceptable salts can be used, such as potassium chloride (KCl), calcium chloride (CaCl₂) and magnesium chloride (MgCl₂) and mixtures thereof, or substances such as propylene glycol, glycerin, dextrose, dextran 40 and 70 or the buffer substances described above.

Antioxidant agents prevent or delay the deterioration of the products resulting from the action of atmospheric oxygen. Among these substances, the most commonly used are ethylenediaminetetraacetic acid (EDTA), thiourea, sodium thiosulfate, sodium metabisulphite and sodium bisulfite.

Preservatives are substances that inhibit bacterial proliferation that can occur after opening the product. Suitable preservatives include for example quaternary ammonium compounds such as benzalkonium chloride, cetyltrimethylammonium bromide and cetylpyridinium chloride, benzethonium hydrochloride, chlorobutanol, EDTA, mercury preservatives (such as thimerosal), phenylethyl alcohol, sodium benzoate, sodium propionate and sorbic acid.

Many of these agents are surfactant compounds which, in addition to inhibiting bacterial proliferation, favor the penetration of drugs through the cornea.

Surfactants have the function of making the composition stable and favoring the penetration of active ingredients into the ocular structures. Examples of surfactants are polysorbates and poloxamers.

In one embodiment, the ophthalmic composition for use according to the invention is an aqueous ophthalmic composition, for example in the form of eye drops for topical administration to the anterior segment of the eye.

The aqueous ophthalmic composition of the TRPA1 channel antagonist comprises water in an amount sufficient to achieve the appropriate concentration of the components of the composition.

Preferably, in the liquid, preferably aqueous, ophthalmic composition, the antagonist of the TRPA1 channel is present in concentrations ranging from about 0.0001% to about 5% w/v, more preferably from about 0.01% to about 1% w/v, even more preferably about 0.5% w/v of the aqueous composition.

An ophthalmic composition for use according to the invention can for example comprise a therapeutically effective amount of at least one TRPA1 channel antagonist compound, sodium chloride, magnesium chloride, mono and di-basic sodium phosphate and water for ophthalmic use to the extent of 100 ml.

In one embodiment, the topical ophthalmic composition, preferably a liquid composition, for use according to the invention can be part of a kit comprising the composition, a container containing the composition and a dispenser. In the case of eye drops, the dispenser is a drop dispenser.

In another embodiment, the pharmaceutical composition, preferably ophthalmic, for use according to the invention can further comprise at least one other pharmaceutically active compound.

In a preferred embodiment, the pharmaceutical composition, preferably ophthalmic, for use according to the invention can further comprise one or more antagonist drugs of vascular endothelial growth factor (anti-VEGF) and/or corticosteroid drugs.

A further aspect of the present description relates to a method for the prevention and/or treatment of at least one degenerative retinal disease, preferably selected from macular degeneration, diabetic retinopathy, retinal detachment, central serous chorioretinopathy, hypertensive retinopathy, macular hole, macular pucker, myodesopsia (floaters) and myopic maculopathy, comprising the administration to a subject of at least one TRPA1 channel antagonist compound or of a pharmaceutical composition, preferably ophthalmic, comprising at least one TRPA1 channel antagonist compound.

In a preferred embodiment, said degenerative retinal disease is macular degeneration and said TRPA1 channel antagonist compound is selected from the compounds belonging to the class of polycyclic derivatives, said compound belonging to the class of polycyclic derivatives being the compound of formula (I), and the compounds belonging to the class of indazole derivatives, said compound belonging to the class of indazole derivatives being the compound of formula (III).

As a guideline, the method according to the invention can comprise the topical ocular administration of 1-100 mg/per administration of at least one TRPA1 channel antagonist, for a daily total of 1-5 administrations.

The exact dose and regimen for the administration of this TRPA1 antagonist in the treatment or prevention of the above-mentioned diseases depend on many factors, such as the route of administration or the degree of distress of the individual receiving the treatment.

In an alternative embodiment, the method comprises administering one or more drugs commonly in use for the treatment of macular degeneration, preferably anti-VEGF drugs and/or corticosteroid drugs, in combination with the TRPA1 channel antagonist compound or with the ophthalmic composition for use according to the invention.

In this embodiment, said drug currently in use for the treatment of macular degeneration, preferably an anti-VEGF drug and/or a corticosteroid drug, can be administered before, during or after the administration of the TRPA1 channel antagonist compound and/or of the ophthalmic composition described above.

Examples of anti-VEGF drugs currently in use for the treatment of macular degeneration that can be administered in combination with the ophthalmic composition comprising at least one TRPA1 channel antagonist compound are ranibizumab, bevacizumab, and/or aflibercept.

Examples of corticosteroid drugs currently in use for the treatment of macular degeneration that can be administered in combination with the ophthalmic composition comprising at least one TRPA1 channel antagonist compound are cortisone, prednisone, prednisolone, methylprednisolone, meprednisone, beclomethasone, triamcinolone, paramethasone, mometasone, budesonide, fluocinonide, halcinonide, flumethasone, flunisolide, fluticasone, betamethasone, dexamethasone, hydrocortisone and/or fluocortolone.

In particular, the method of prevention and/or treatment of a degenerative retinal disease includes the administration of at least one of the corticosteroid drugs listed above when said degenerative retinal disease is selected from diabetic retinopathy, retinal detachment, central serous chorioretinopathy, hypertensive retinopathy.

According to the present invention, the administration of the anti-VEGF drug and/or corticosteroid drug can occur simultaneously, separately or sequentially.

In another embodiment, the method for the prevention and/or treatment of at least one degenerative retinal disease as previously defined, comprises administering to a subject a composition comprising at least one TRPA1 channel antagonist compound and at least one drug selected from anti-VEGF drugs and corticosteroid drugs.

In said embodiment, the drug currently in use for the treatment of retinal diseases, for example macular degeneration, is already included in the composition of the invention and is therefore co-administered with the TRPA1 channel antagonist compound and/or the composition comprising said compound.

A final aspect of the present invention relates to a combination of at least one TRPA1 channel antagonist compound and an anti-VEGF drug and/or a corticosteroid drug for simultaneous, separate or sequential use in the prevention and/or treatment of at least one degenerative retinal disease selected from macular degeneration, diabetic retinopathy, retinal detachment, central serous chorioretinopathy, hypertensive retinopathy, macular hole, macular pucker, myodesopsia (floaters) and myopic maculopathy, preferably macular degeneration in both its forms.

Preferably the combination for use according to the invention comprises as a TRPA1 channel antagonist compound at least one compound belonging to classes 2, 5) and 6) as defined above, more preferably a compound of formula I-IV according to the present description.

In a preferred embodiment, said combination is useful for the prevention and/or treatment of macular degeneration, in particular dry age-related macular degeneration and wet age-related macular degeneration.

In said embodiment, the combination comprises an anti-VEGF drug selected from ranibizumab, bevacizumab, or aflibercept.

In said embodiment, the combination comprises a corticosteroid drug selected from cortisone, prednisone, prednisolone, methylprednisolone, meprednisone, beclomethasone, triamcinolone, paramethasone, mometasone, budesonide, fluocinonide, halcinonide, flumethasone, flunisolide, fluticasone, betamethasone, dexamethasone, hydrocortisone and fluocortolone.

### DESCRIPTION OF THE FIGURES

FIG. 1A shows immunofluorescence images of the RPE layer in the retina collected on day 4 from mice in the NalO₃-induced macular degeneration model, after the treatment with NalO₃ or its vehicle (V1) and treated with:
compound of formula (IV) (indicated as FN-005)—class 2) sulfonamide derivatives; or
compound of formula (I) (indicated as FN-006)—class 5) heteroaromatic polycyclic derivatives; or
compound of formula (II) (indicated as FN-007)—class 5) heteroaromatic polycyclic derivatives; or
compound of formula (III) (indicated as FN-008)—class 6) indazole derivatives and bioisosteres;
or their vehicle (V2).

RPE65 indicates the antibody used for staining the RPE layer.

The writings shown on each panel of FIG. 1A indicate:
V1/V2: the mice were administered the vehicle (V1) wherein NalO₃ is dissolved and before and after they received the administration of the vehicle wherein the various drugs are dissolved (V2), and thus they did not receive any active treatment. The V2 vehicle wherein the various drugs were dissolved consisted of 4% dimethyl sulfoxide (DMSO), 4% Tween 80 in 0.9% NaCl.
V2/NalO₃: the mice were administered the vehicle wherein the various drugs are dissolved (V2) before and after the administration of NalO₃ dissolved in its vehicle (V1) to obtain mice with macular degeneration.
FN-005/NalO₃: the mice were treated with the compound of formula (IV) before and after the administration of NalO₃ dissolved in its vehicle.
FN-006/NalO₃: the mice were treated with the compound of formula (I) before and after the administration of NalO₃ dissolved in its vehicle.
FN-007/NalO₃: the mice were treated with the compound of formula (II) before and after the administration of NalO₃ dissolved in its vehicle.
FN-008/NalO₃: the mice were treated with the compound of formula (III) before and after the administration of NalO₃ dissolved in its vehicle.

FIG. 1B is a histogram representing the cumulative data of the immunofluorescence experiment shown in FIG. 1A.

FIG. 2A shows immunofluorescence images of the oxidative stress biomarker 4-hydroxynonenal (4-HNE) in the retina collected on day 4 from mice in the model of macular degeneration induced by NalO₃, after the treatment with NalO₃ or its vehicle (V1) and treated with:
compound of formula (IV) (indicated as FN-005)—class 2) sulfonamide derivatives; or
compound of formula (I) (indicated as FN-006)—class 5) heteroaromatic polycyclic derivatives; or
compound of formula (II) (indicated as FN-007)—class 5) polycyclic heteroaromatic derivatives; or
compound of formula (III) (indicated as FN-008)—class 6) of indazole derivatives and bioisosteres;
or their vehicle (V2).

The writings shown on each panel of FIG. 2A indicate:
V1/V2: the mice were administered the vehicle wherein the various drugs are dissolved (V1) and before and after they received the administration of the vehicle (V2) wherein NalO₃ is dissolved. These mice constitute the controls.
V2/NalO₃: the mice were administered the vehicle (V2) wherein the various drugs are dissolved before and after the administration of NalO₃ dissolved in its vehicle to obtain mice with macular degeneration.
FN-005/NalO₃: the mice were treated with the compound of formula (IV) before and after the administration of NalO₃ dissolved in its vehicle.
FN-006/NalO₃: the mice were treated with the compound of formula (I) before and after the administration of NalO₃ dissolved in its vehicle.
FN-007/NalO₃: the mice were treated with the compound of formula (II) before and after the administration of NalO₃ dissolved in its vehicle.
FN-008/NalO₃: the mice were treated with the compound of formula (II) before and after the administration of NalO₃ dissolved in its vehicle.

FIG. 2B is a histogram representing the cumulative data of the immunofluorescence experiment shown in FIG. 2A.

### EXAMPLES

### Example 1—Mouse Model of Macular Degeneration

In order to test the compounds for use according to the invention, a mouse model of macular degeneration was obtained.

The in vivo experiments were performed in compliance with the Italian legislation (Legislative Decree 26/2014) and the guidelines provided by the European regulation (EU Directive 2010/63/EU). The study was conducted after the approval of the protocol by the Ministry of Health (protocol no 135/2022-PR).

To create a valid model of macular degeneration, the systemic administration (via the retro-orbital vein) of NalO₃ was carried out in C57BL/6J male mice aged 5-8 weeks and weighing 22-25 g supplied by the Charles River company (Milan, Italy). In order to carry out the experiments described below, a total of 36 mice were used. The animals were kept in a temperature and humidity-controlled environment (12-hour dark/light cycle, free access to food and water). The experiments were performed in a temperature-controlled room (20 to 22° C.) between 8:00 and 20:00. At the end of the experiment, the animals were euthanized by inhalation of a mixture of 50% O₂/50% CO₂ for 1 minute. The NalO₃, DMSO, Tween 80 and NaCl 0.9% reagents used in the study were purchased from Merck Life Science SRL (Milan, Italy).

The compounds tested in example 2 (compound belonging to class 2) of the sulfonamide derivatives having formula (IV) (GDC-0334) and hereinafter referred to as FN-005; compound belonging to class 5) of polycyclic heteroaromatic derivatives having formula (I) (compound 10, Amgen) and hereinafter referred to as FN-006; compound belonging to class 5) of polycyclic heteroaromatic derivatives having formula (II) (compound 43, Janssen) and hereinafter referred to as FN-007; and compound belonging to class 6) of indazole derivatives and bioisosteres having formula (III) (compound 31, Novartis) and hereinafter referred to as (FN-008)] have been synthesized according to processes known in the art.

The mouse model obtained by systemic administration (via the retro-orbital vein) of NalO₃ is a model of macular degeneration.

In fact, it was observed that 3 days after administration, NalO₃ induced persistent retinal damage in the mice subjected to the experiments with similar characteristics to those observed in age-related macular degeneration in humans.

### Example 2—Administration of Preferred Compounds Belonging to Classes 2), 5) and 6) as Indicated in the Present Description

In order to assess the efficacy of preferred compounds belonging to classes 2), 5) and 6) according to the present description in reducing and treating the retinal damage typical of macular degeneration, an experiment was set up on 6 groups of model mice, obtained according to the procedure described in Example 1.

In particular, the following compounds were tested:
compound belonging to class 2) of sulfonamide derivatives having formula (IV) (GDC-0334) and hereinafter referred to as FN-005;
compound belonging to class 5) of polycyclic heteroaromatic derivatives having formula (I) (compound 10, Amgen) and hereinafter referred to as FN-006;
compound belonging to class 5) of polycyclic heteroaromatic derivatives having formula (II) (compound 43, Janssen) and hereinafter referred to as FN-007; and
compound belonging to class 6) of indazole derivatives and bioisosteres having formula (III) (compound 31, Novartis) and hereinafter referred to as (FN-008).

The mice were administered locally, through eye drops, the compounds indicated above or their vehicle consisting of 4% DMSO, 4% Tween 80 in 0.9% NaCl.

In particular, a group of 6 mice (used as a control) was administered by instillation, 60 minutes before the injection into the retro-orbital vein (1 ml/kg) of the vehicle (V1) (NaCl, 0.9%) of NalO₃ and subsequently 3 times a day, eye drops (5 μl) containing the vehicle (V2) (4% DMSO, 4% Tween 80 in NaCl 0.9%) of the drugs.

Another 30 mice received, in groups of 6 mice each, 60 minutes before the injection into the retro-orbital vein (1 ml/kg) of NalO₃ (1%, 20 mg/kg) and subsequently 3 times a day, eye drops (5 μl) of a 10 mM solution of the above-mentioned compounds FN-005, FN-006, FN-007, FN-008 or their vehicle (V2) (4% DMSO, 4% Tween 80 in 0.9% NaCl) by instillation.

For each group of mice, the first administration (day 1) of the compound of formula (IV), of the compound of formula (I), of the compound of formula (II) and of the compound of formula (III) or of the vehicle (V2) was performed 60 minutes before the injection of NalO₃ or its vehicle (V1) and the second and third administration were performed 6 and 12 hours after the injection of vehicle (V1) or NalO₃, respectively. On the two days (day 2 and day 3) following the injection of vehicle (V1) or NalO₃, the compounds of formula (IV), (I), (II), and (III) or vehicle (V2) were administered to the various groups of mice at 8:00, 14:00 and 20:00.

At 09:00 on day 4 after the treatment with vehicle (V1) or NalO₃ the mice given the compounds indicated above as FN-005, FN-006, FN-007, FN-008 or their vehicle (V2) were sacrificed (as previously reported) and the eyeballs were enucleated and processed for subsequent analysis of the damage.

### Example 3—Immunofluorescence for Assessing the Damage of the Retinal Pigment Epithelium

Direct immunofluorescence was used to assess damage to the RPE (retinal pigmented epithelium), which corresponds to the layer of pigmented cells immediately adjacent to the neurosensory retina which nourishes the visual cells of the retina, and is firmly attached to the underlying choroid and the overlying visual retinal cells.

The staining intensity of the RPE layer was quantified using a primary antibody (RPE65, #ab13826, mouse monoclonal, 1:100, Abcam, Cambridge, UK) to which a second fluorophore-labelled antibody (Alexa Fluor 488, #A28175, Thermo Fisher Scientific) binds in the 6 groups of mice treated with V2/V1, V2/NalO₃, with the compound of formula (IV) and NalO₃ (FN-005/NalO₃), with the compound of formula (I) of polycyclic heteroaromatic derivatives and NalO₃ (FN-006/NalO₃), with the compound of formula (II) and NalO₃ (FN-007/NalO₃) and with the compound of formula (III) and NalO₃ (FN-008/NalO₃). Cell nuclei were visualized using the DAPI organic dye (#ab228549, Abcam, Cambridge, UK).

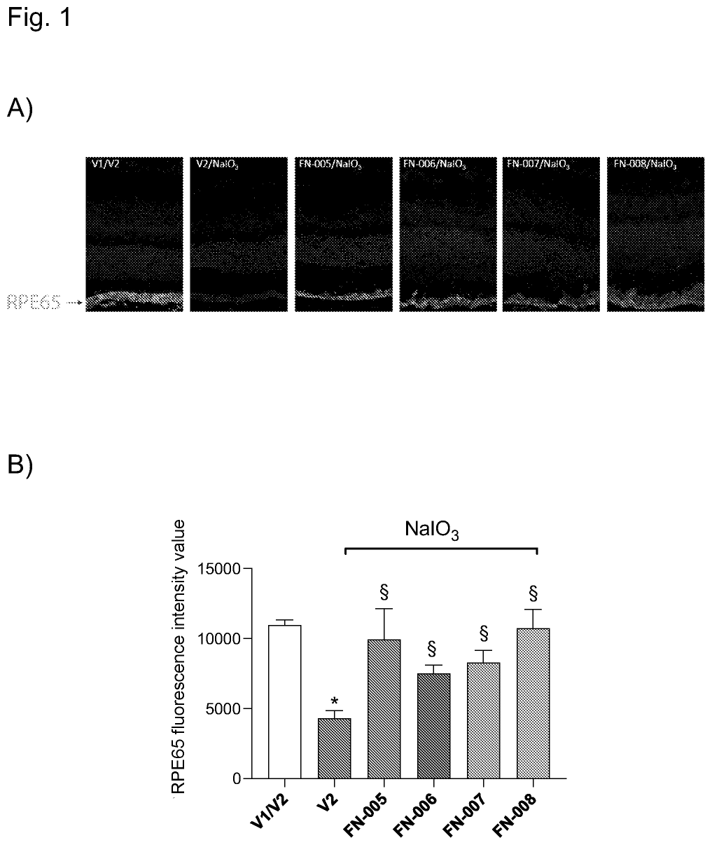

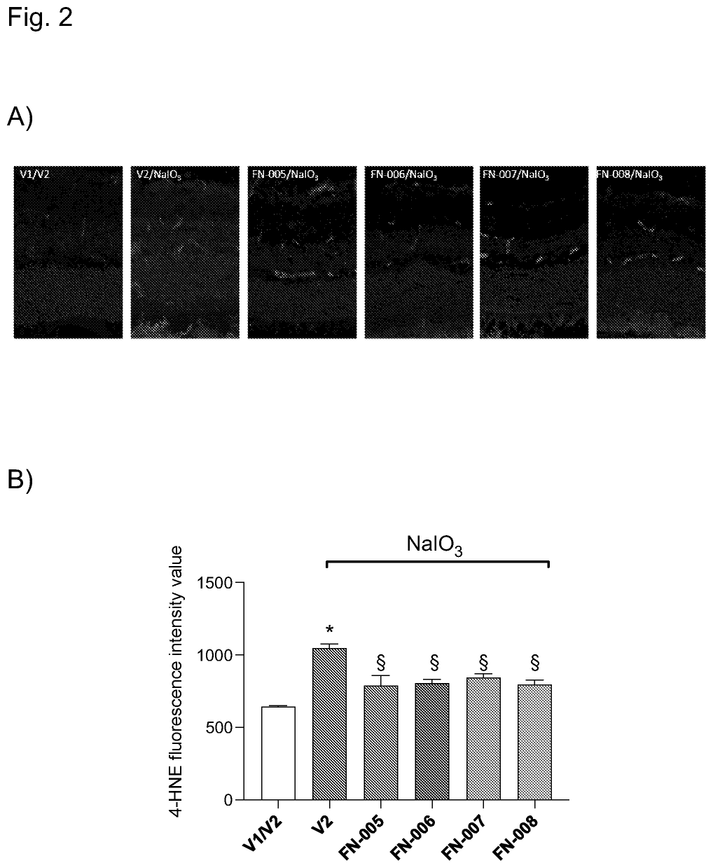

FIGS. 1A and 1B show representative images and cumulative data of the immunofluorescent staining of the RPE layer, performed using a primary antibody (RPE65), in the retina collected on day 4 in the 6 groups of mice treated with V2/V1, V2/NalO₃, with the compound of formula (IV) and NalO₃ (FN-005/NalO₃), with the compound of formula (I) and NalO₃ (FN-006/NalO₃), with the compound of formula (II) and NalO₃ (FN-007/NalO₃) and with the compound of formula (III) and NalO₃ (FN-008/NalO₃).

The treatments indicated above are shown in FIG. 1A as: V2/V1, V2/NalO₃, FN-005/NalO₃, FN-006/NalO₃, FN-007/NalO₃ and FN-008/NalO₃.

In mice that received the injection of NalO₃, a reduction in the intensity of the staining of the RPE layer of 60.7±5.00% (P<0.01 vs. V1/V2) was observed (FIGS. 1A and 1B). Treatment with eye drops with the compound of formula (IV), the compound of formula (I), the compound of formula (II) and the compound of formula (III) statistically significantly reduced the NalO₃-induced damage on the RPE layer compared to V2 by 84.5±33% (P<0.01 vs. V2/NalO₃), 48.0±9.1% (P<0.01 vs. V2/NalO₃), 60.0±12.8% (P<0.01 vs. V2/NalO₃) and 96.8±20.0% (P<0.01 vs. V2/NalO₃) respectively (FIGS. 1A and 1B).

The data relating to the fluorescence intensity value are presented for each treatment as mean±SEM. *p<0.05 vs V1/V2; § p<0.05 vs NalO₃ in FIG. 1B. Statistical analysis using one-way analysis of variance (ANOVA) test and Bonferroni test.

### Example 4—Assessment of Oxidative Stress at the Retinal Level

The level of oxidative stress was also assessed throughout the thickness of the retina by measuring the immunofluorescence intensity for the reactive carbonyl species, 4-hydroxynonenal (4-HNE), a final indicator of oxidative stress.

Levels of 4-HNE were quantified using a primary antibody (#ab48506, monoclonal mouse [HNEJ-2], 1:40, Abcam, Cambridge, UK) to which a second fluorophore-labelled antibody (Alexa Fluor 594, #A A32742, Thermo Fisher Scientific) binds in the 6 groups of mice treated as described above.

Cell nuclei were visualized using the DAPI organic dye (#ab228549, Abcam, Cambridge, UK).

FIGS. 2A and 2B show representative images and cumulative data of the immunofluorescence staining of the oxidative stress biomarker 4-HNE in the 6 groups of mice treated with V2/V1, V2/NalO₃, with the compound of formula (IV) and NalO₃ (FN-005/NalO₃), with the compound of formula (I) and NalO₃ (FN-006/NalO₃), with the compound of formula (II) and NalO₃ (FN-007/NalO₃) and with the compound of formula (III) and NalO₃ (FN-008/NalO₃).

The treatments indicated above are shown in FIG. 2A as: V2/V1, V2/NalO₃, FN-005/NalO₃, FN-006/NalO₃, FN-007/NalO₃ and FN-008/NalO₃.

The administration of NalO₃ induced a 62.89±4.20% (P<0.001 vs. V1/V2) increase in 4-HNE immunofluorescence throughout the retinal tissue (FIGS. 2A and 2B).

The treatment with the compound of formula (IV), compound of formula (I), compound of formula (II) and compound of formula (III) statistically significantly reduced the retinal levels of 4-HNE respectively by 64.16±17.51% (P<0.01 vs. V2/NalO₃), 59.56±6.31% (P<0.01 vs. V2/NalO₃), 50.32±6.30% (P<0.01 vs. V2/NalO₃), 62.10±7.55% (P<0.01 vs. V2/NalO₃) (FIGS. 2A and 2B).

The data relating to the fluorescence intensity value are presented for each treatment as mean±SEM. *p<0.05 vs V1/V2; § p<0.05 vs NalO₃ in FIG. 2B. Statistical analysis using one-way analysis of variance (ANOVA) test and Bonferroni test.

From the experimental evidence, it is therefore possible to conclude that the tested compounds perform a protective action on the NalO₃-induced damage of the cells of the RPE layer which is of fundamental importance for the maintenance of the photoreceptor function of the macula. Furthermore, it was observed that the tested compounds, having formula (IV), (I), (II) and (III), protected the retina from the NalO₃-induced increase of 4-HNE.

1: A method of preventing and/or treating a degenerative retinal disease which comprises administering to a patient in need thereof an effective amount of a TRPA1 channel antagonist compound, wherein the degenerative retinal disease is selected from the group consisting of macular degeneration, diabetic retinopathy, retinal detachment, central serous chorioretinopathy, hypertensive retinopathy, macular hole, macular pucker, myodesopsia (floaters) and myopic maculopathy.

2-4. (canceled)

5: The method according to claim 1, wherein the TRPA1 channel antagonist compound belongs to one of the following classes:
2) sulfonamide derivatives;
5) polycyclic heteroaromatic derivatives; or
6) indazole derivatives and bioisosteres,
the salts thereof, optical isomers, solvates and prodrugs.

6: The method according to claim 5, wherein the compound belonging to class 2) sulfonamide derivatives has general formula A1: 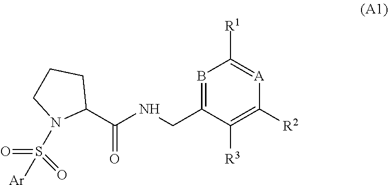
where A and B, equal or different from each other, can represent a CH group or a nitrogen atom, Ar can represent a 5 or 6-membered aromatic cycle, preferably selected from the group consisting of aryl, pyridine, pyrimidine, pyrazine, pyrrole, imidazole, furan, thiophene, and thiazole, optionally substituted with one or more halogen atoms, preferably with one or more fluorine or chlorine atoms, and R1, R2 and R3 equal or different from each other can represent a hydrogen atom, a fluoromethyl group, or the residue of formula: 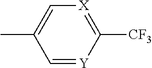
where X and Y, equal or different from each other, can represent a CH group or a nitrogen atom.

7: The method according to claim 5, wherein the compound belonging to the class 2) sulfonamide derivatives is the compound of formula (IV):

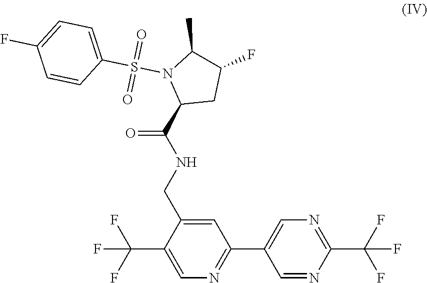 [SMILES: C[C@H]1[C@H](F)C[C@@H](C(=O)NCc2cc(-c3cnc(C(F)(F)F)nc3)ncc2C(F)(F)F)N1S(=O)(=O)c1ccc(F)cc1]

8: The method according to claim 5, wherein the compound belonging to class 5) polycyclic heteroaromatics derivatives has general formula A2: 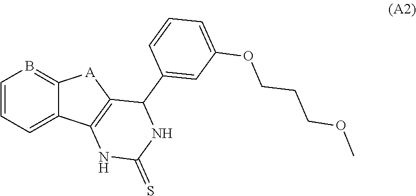
where A can represent an oxygen atom, a —NH— group, or a carbonyl group —(C═O)—, and B can represent a —CH— group or a nitrogen atom.

9: The method according to claim 5, wherein the compound belonging to class 5) polycyclic heteroaromatic derivatives is the compound of formula (I) or the compound of formula (II):

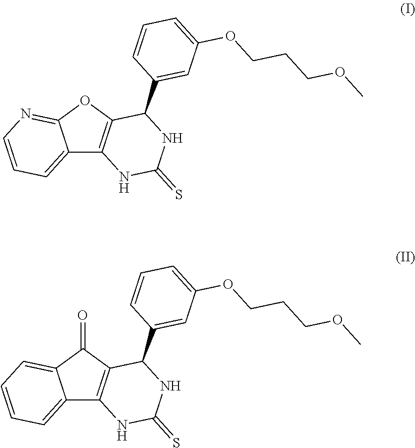 [SMILES: COCCCOc1cccc([C@H]2NC(=S)Nc3c2oc2ncccc32)c1 ; COCCCOc1cccc([C@H]2NC(=S)NC3=C2C(=O)c2ccccc23)c1]

10: The method according to claim 5, wherein the compound belonging to class 6) indazole derivatives and bioisosteres has general formula A3: 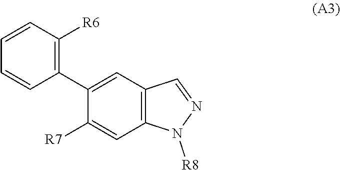
where R6, R7 and R8, equal or different from each other, can represent a hydrogen atom, an alkyl group having from 1 to 3 carbon atoms, or a trifluoromethyl group.

11: The method according to claim 5, wherein the compound belonging to class 6) indazole derivatives and bioisosteres is the compound of formula (III):

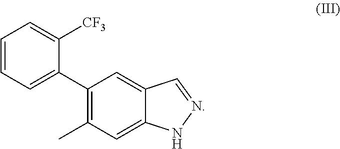 [SMILES: Cc1cc2[nH]ncc2cc1-c1ccccc1C(F)(F)F]

12: A method of preventing and/or treating a degenerative retinal disease which comprises administering to a patient in need thereof an effective amount of a pharmaceutical composition comprising at least one TRPA1 channel antagonist compound and at least one pharmaceutically acceptable excipient, wherein the degenerative retinal disease is selected from the group consisting of macular degeneration, diabetic retinopathy, retinal detachment, central serous chorioretinopathy, hypertensive retinopathy, macular hole, macular pucker, myodesospia (floaters) and myopic maculopathy.

13: The method according to claim 12, wherein the at least one TRPA1 channel antagonist compound belongs to one of the following classes:
2) sulfonamide derivatives;
5) polycyclic heteroaromatic derivatives; or
6) indazole derivatives and bioisosteres,
the salts thereof, optical isomers, solvates and prodrugs.

14: The method according to claim 12, wherein the compound belonging to class 2) sulfonamide derivatives has general formula A1: 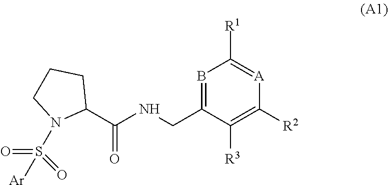
where A and B, equal or different from each other, can represent a CH group or a nitrogen atom, Ar can represent a 5 or 6-membered aromatic cycle, preferably selected from the group consisting of aryl, pyridine, pyrimidine, pyrazine, pyrrole, imidazole, furan, thiophene, and thiazole, optionally substituted with one or more halogen atoms, preferably with one or more fluorine or chlorine atoms, and R1, R2 and R3 equal or different from each other can represent a hydrogen atom, a fluoromethyl group, or the residue of formula: 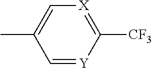
where X and Y, equal or different from each other, can represent a CH group or a nitrogen atom.

15: The method according to claim 14, wherein the compound belonging to class 2) of sulfonamide derivatives is the compound of formula (IV):

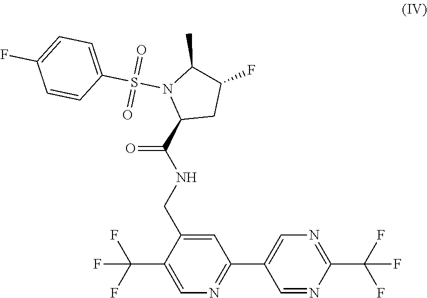 [SMILES: C[C@H]1[C@H](F)C[C@@H](C(=O)NCc2cc(-c3cnc(C(F)(F)F)nc3)ncc2C(F)(F)F)N1S(=O)(=O)c1ccc(F)cc1]

16: The method according to claim 12, wherein the compound belonging to class 5) polycyclic heteroaromatics derivatives has general formula A2: 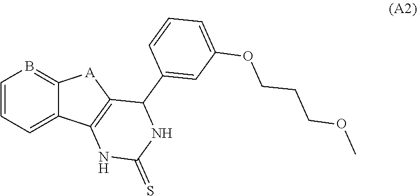
where A can represent an oxygen atom, a —NH— group, or a carbonyl group —(C═O)—, and B can represent a —CH— group or a nitrogen atom.

17: The method according to claim 13, wherein the compound belonging to class 5) polycyclic heteroaromatic derivatives is the compound of formula (I) or the compound of formula (II):

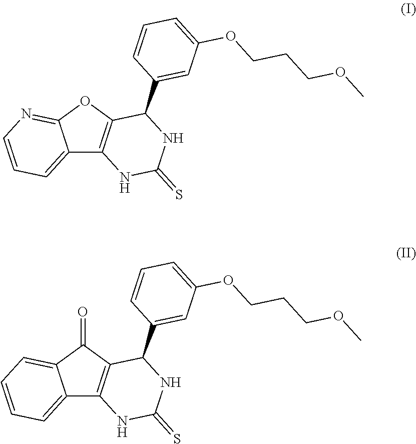 [SMILES: COCCCOc1cccc([C@H]2NC(=S)Nc3c2oc2ncccc32)c1 ; COCCCOc1cccc([C@H]2NC(=S)NC3=C2C(=O)c2ccccc23)c1]

18: The method according to claim 12, wherein the compound belonging to class 6) indazole derivatives and bioisosteres has general formula A3: 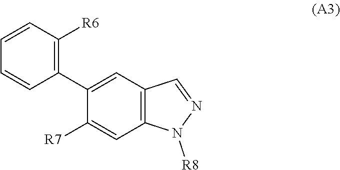
where R6, R7 and R8, equal or different from each other, can represent a hydrogen atom, an alkyl group having from 1 to 3 carbon atoms, or a trifluoromethyl group.

19: The method according to claim 13, wherein the compound belonging to class 6) indazole derivatives and bioisosteres is the compound of formula (III):

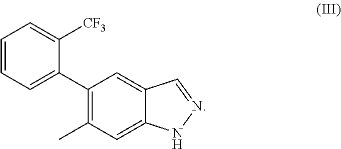 [SMILES: Cc1cc2[nH]ncc2cc1-c1ccccc1C(F)(F)F]

20-22. (canceled)

23: The method according to claim 12, wherein the composition is an ophthalmic composition comprising the at least one TRPA1 channel antagonist compound and at least one ophthalmologically acceptable excipient.

24: The method according to claim 23, wherein the ophthalmic composition is a topical ophthalmic composition.

25-27. (canceled)

28: A kit comprising a topical ophthalmic composition, a container containing it and a dispenser, wherein the composition comprises the at least one TRPA1 channel antagonist compound and at least one pharmaceutically acceptable excipient as defined in claim 12.

29: A method of preventing and/or treating a degenerative retinal disease which comprises administering to a patient in need thereof an effective amount of a combination of a TRPA1 channel antagonist compound and an anti-VEGF drug and/or a corticosteroid drug, wherein the degenerative retinal disease is selected from the group consisting of macular degeneration, diabetic retinopathy, retinal detachment, central serous chorioretinopathy, hypertensive retinopathy, macular hole, macular pucker, myodesopsia (floaters) and myopic maculopathy.

30. (canceled)

31: The method according to claim 29, wherein the anti-VEGF drug is selected from the group consisting of ranibizumab, bevacizumab, and/or aflibercept.

32: The method according to claim 29, wherein the corticosteroid drug is selected from the group consisting of cortisone, prednisone, prednisolone, methylprednisolone, meprednisone, beclomethasone, triamcinolone, paramethasone, mometasone, budesonide, fluocinonide, halcinonide, flumethasone, flunisolide, fluticasone, betamethasone, dexamethasone, hydrocortisone and/or fluocortolone.
